(19) United States
(12) Patent Application Publication
(10) Pub. No.: US 2025/0025590 A1
(43) Pub. Date: Jan. 23, 2025

(54) Electrolytic Ozone Cleaner and Connector for Aqueous-Circuit Medical Devices

(71) Applicant: Medical Innovation Solutions and Technologies, LLC, Delray Beach, FL (US)

(72) Inventors: William Dolan, Delray Beach, FL (US); Kory Wagner, Delray Beach, FL (US)

(73) Assignee: Medical Innovation Solutions and Technologies, LLC, Delray Beach, FL (US)

(21) Appl. No.: 18/774,972

(22) Filed: Jul. 17, 2024

Related U.S. Application Data
(60) Provisional application No. 63/513,919, filed on Jul. 17, 2023.

(51) Int. Cl. A61L 2/18; A61B 90/70; A61L 2/26; A61L 101/02

(52) U.S. Cl. CPC A61L 2/183; A61B 90/70; A61L 2/26; A61L 2101/02; A61L 2202/11; A61L 2202/121; A61L 2202/123; A61L 2202/14; A61L 2202/16; A61L 2202/17; A61L 2202/24

(57) ABSTRACT

An ozone cleaner and connector for aqueous-circuit medical devices comprises an exterior housing and a connection for an ozone generator for use in cleaning and/or disinfecting mobile medical devices containing aqueous solution and circuit design. The ozone cleaner and connector lessen the need for chemicals and harmful exposure, and the ozone cleaner and connector provide an easier and more time-efficient cleaning procedure.

### CROSS REFERENCE TO RELATED APPLICATION

This application is based upon and claims priority to U.S. provisional application No. 63/513,919, filed on Jul. 17, 2023, the entire content of which is incorporated herein by reference.

### TECHNICAL FIELD

The ozone cleaner and connector for aqueous-circuit medical devices relates to electrolytic and venturi ozone generators that are used in the field of water treatment; this disclosure aims to revise the standard cleaning procedure of aqueous-circuit medical devices and mobile medical devices that contain aqueous or water solutions.

### BACKGROUND

Ozone has been used for water treatment and disinfection for many years. This chemical process is achieved by running an electrical current through a special type of electrode typically made of boron doped diamond or one of ceramic plate design. This electrochemical reaction creates ozonated water directly using only the molecules in the water and air to do so. The process creates aqueous ozone, a strong sanitizing and disinfecting agent due to its oxidative properties. Through the ceramic plate design this process is achieved by a corona discharge method and injected and mixed into the water or aqueous solution by use of a venturi gas injection. Aqueous ozone has also been shown to be able to reduce most bacteria at a much quicker rate than commercial chemicals and has also been shown to reduce most biofilms to a greater degree. Although ozone in its gaseous form is hazardous, when ozone is produced either by an electrochemical process or through direct venturi injection into water there are negligible toxic off gases produced. In addition to the aqueous form of ozone being non-hazardous it also has a short half-life when produced in aqueous solutions and will dissipate usually within an hour leaving no chemical residue. New adaptations have recently been made allowing the ozone generation technology to be integrated into smaller aqueous systems. The current standard methods of cleaning and disinfecting aqueous-circuit medical devices is through a chemical additive process with multiple stages of filling and emptying to ensure no harmful chemical residue remains. These aqueous-circuit medical devices have come under scrutiny for inadequate cleaning procedures and lack of biofilm reduction with the chemical cleaning processes they use. FIG. 1 represents an example of a prior aqueous-circuit medical device and its hose line connections.

### SUMMARY

The ozone cleaner and connector for aqueous-circuit medical devices would reduce the need for chemical additives, eliminating waste and harmful exposure for the staff involved. The present invention would also reduce the amount of time and steps required to complete the cleaning procedure required for the aqueous-circuit medical devices. Aqueous ozone's half-life eliminates the need to drain the machine after cleaning since the chemical constituents will return to their normal state within a short time frame. Depending on the storage location of the aqueous-circuit medical devices, it will also eliminate the need to move the machines to a location with a drain. Additionally, the ozone cleaner and connector for aqueous-circuit medical devices can be used as a connection to keep the aqueous-circuit medical devices lines from dripping and being exposed to open air when not being cleaned and when not in use. This would also provide a storage solution for the ozone cleaner and connector for aqueous-circuit medical devices.

### BRIEF DESCRIPTION OF THE DRAWINGS

FIG. 1 represents an example of a prior aqueous-circuit medical device and hose line connections.

FIG. 2 illustrates a first perspective view of the housing apparatus of the present disclosure and the quick connection of the housing apparatus.

FIG. 3 illustrates a second perspective view of the housing apparatus and the quick connection of the housing connections.

FIG. 4 depicts an inner view of the two-piece housing opened, including internal hoses, quick connections to the ozone generator, and an internal processor for the digital display.

FIG. 5 depicts the quick connection including male to female hose connections on the ozone cleaner and connector for aqueous-circuit medical devices to be used to connect to the aqueous medical device circuit hose lines.

FIG. 6 depicts an example of an aqueous-circuit medical device with a pair of hose lines connected to the ozone cleaner and connector, creating a continuous flow throughout the medical device and the aqueous ozone generator housed and connected within the housing apparatus.

FIG. 7 is a detailed illustration of the PCB comprised in the housing apparatus.

FIG. 8 illustrates components and construction of the ½ inch to ⅜ inch threading converter with ball valve and honeycomb diffusion disk.

FIG. 9 illustrates the structure of the ½ inch to ⅜ inch threading converter with ball valve and honeycomb diffusion disk.

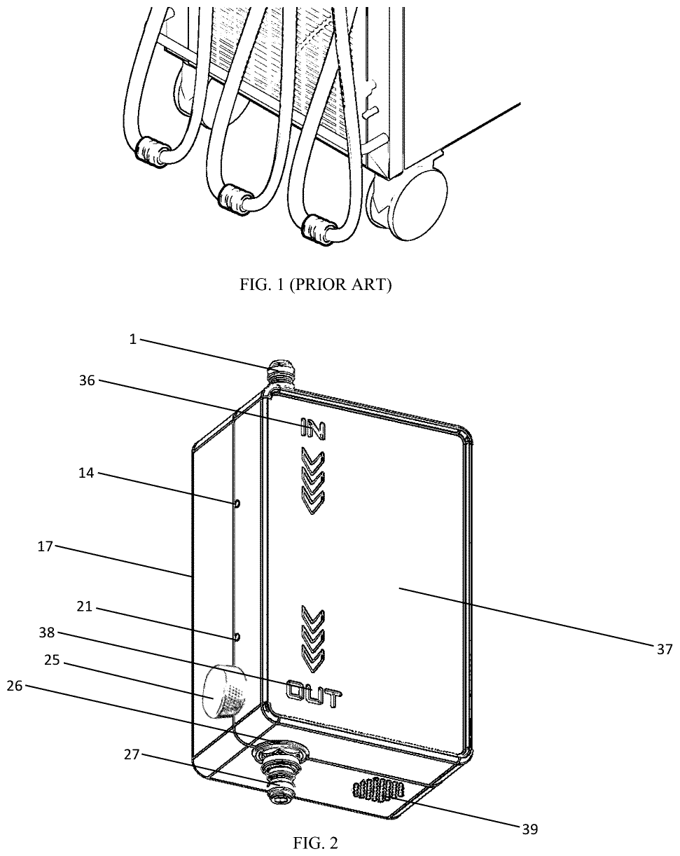

### REFERENCES

1: first Hansen B3-T20⅜ inch male NPTF quick-connect, 2: first rubber grommet, 3: first power input screw point, 4: second power input screw point, 5: power input for external power source, 6: first PCB and PCB cover screw point, 7: second PCB and PCB cover screw point, 8: ½ inch to ⅜ inch threading converter, 9: PCB with protective cover, 10: power toggle on/off switch and/or button, 11: first device housing screw point, 12: ½ inch venturi injector inline component with built-in one-way valve for aqueous ozone solution, 13: ¼ inch silicon tubing, 14: second device housing screw point, 15: third PCB and PCB cover screw point, 16: fourth PCB and PCB cover screw point, 17: ozone device housing, 18: ½ inch female-to-female circuit fitting, 19: ozone generator with cooling fan and heat-sink housing, 20: third device housing screw point, 21: fourth device housing screw point, 22: ½ inch turbine flow sensor, 23: ½ inch to ⅜ inch threading converter with ball valve and honeycomb diffusion disk, 24: valve stem with seals, 25: indicator knob, 26: second rubber grommet, 27: second Hansen B3-T20⅜ inch male NPTF quick-connect, 28: ½ inch BSP female threading inflow, 29: O-ring lip inside ½ inch BSP female threading, 30: honeycomb diffusion disk, 31: first gasket, 32: ball valve, 33: second gasket, 34: grommet lock, 35: ⅜ inch NPTF threading conversion, 36: embossed front plate “In” with arrows, 37: front face plate, 38: embossed front plate “Out” with arrows, 39: Air intake vent, 40: Power toggle on/off switch and/or button, 41: LED Strip, 42: Power on/off, 43: Alarm, 44: Flow sensor, 45: Power cord outlet, 46: Ozone generator controller.

### Detailed Description of the Embodiments

The ozone cleaner and connector for aqueous-circuit medical devices will improve the cleaning process for the aqueous-circuit and mobile medical devices by using an ozone generator that will be designed to connect to the water line connections. The connections inside the ozone cleaner and connector for aqueous-circuit medical devices will have an adaptive and quick connection design that will allow for connection to a range of different ozone generators. The ozone cleaner and connector for aqueous-circuit medical devices will allow the flow of water and other aqueous solutions through the ozone generator creating aqueous ozone as a disinfecting and/or sanitizing solution in the device. The present invention will be connected in a way that adds to the continuous circuit of the aqueous flow throughout the medical devices. This continuous flow design will allow the aqueous ozone produced from the ozone generator to circulate through the medical devices, the housing and connection lines in the present invention, and the ozone generator connected inside for the desired set time to reach adequate cleaning standards.

The ozone cleaner and connector for aqueous-circuit medical devices will look to replace and/or add to current regulatory cleaning methods used with circuit and mobile aqueous containing medical devices. The present invention will alleviate the amount of time and effort required to clean these medical devices by shortening the overall time for the cleaning process as well as eliminating many steps in the process such as filling and draining the devices multiple times. This will be achieved in part by aqueous ozone's short half-life in aqueous solutions that allows it to dissipate and return the solution to its prior chemical make-up. This will also be achieved by the present invention's easy quick connection design that will allow it to connect directly to the aqueous medical devices circuit lines.

The present invention will also eliminate the time needed to measure out chemical additives since its cleaning method will be achieved by connecting to the aqueous-circuit medical devices and then powering the ozone generator on through the housing apparatus. The present invention aims to eliminate chemical and plastic waste from the current cleaning procedure while also saving time and labor required by the professional staff involved.

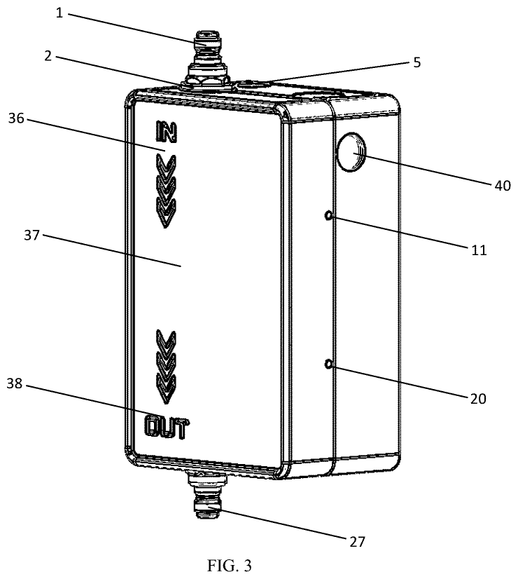

FIGS. 2 and 3 illustrate the housing apparatus of the present disclosure and the quick connection of the housing apparatus. Male hose quick connectors 1 and 27 will be on both ends of the ozone cleaner and connector for aqueous-circuit medical devices allowing for quick connection and flow throughout the invention and the ozone generator housed within. The output display as depicted below will give readout of water metrics and ozone production and will have the ability to show a timer countdown linked to an automatic power down of ozone generator when timer reaches zero. This timer will be set and altered according to cleaning specifications and results and will be set using the buttons as shown in the depictions.

As illustrated in FIG. 2, the housing apparatus comprises a Hansen B3-T20⅜ inch male NPTF quick-connect 1 located at the water inflow port for connection to female end of aqueous-circuit medical devices, an embossed front plate “In” with arrows 36 and an embossed front plate “Out” with arrows 38 signify clearly the direction of water flow through the device, device housing screw points 14 and 21 each allows a short screw to connect top and bottom shell of the device housing, front face plate 37 fitted onto bottom house and secured by device housing screw points 11, 14, 20, 21, an indicator knob 25 that allows movement of ball valve from outside of the unit, rubber grommet 26 located at the water outflow port between quick-connect male end and device internal water circuit, which acts as a seal between the environment and the device internal while stabilizing the internal circuit, a Hansen B3-T20⅜ inch male NPTF quick-connect 27 located at the water outflow port for connection to female end of aqueous-circuit medical devices, and an air intake vent 39 that allows air to be directly pulled into space within device as ozone generator coupled with venturi injector force air into the internal circuit and out of the device.

As shown in FIG. 3, the housing apparatus comprises a Hansen B3-T20⅜ inch male NPTF quick-connect 1 located at the water inflow port for connection to female end of aqueous-circuit medical devices, an embossed front plate “In” with arrows 36 and an embossed front plate “Out” with arrows 38 signify clearly the direction of water flow through the device, device housing screw points 11 and 20 each allows a short screw to connect top and bottom shell of the device housing, front face plate 37 fitted onto bottom house and secured by device housing screw points 11, 14, 20, 21, rubber grommet 2 located at the water inflow port between quick-connect male end and device internal water circuit, which acts as a seal between the environment and the device internal while stabilizing the internal circuit, a Hansen B3-T20⅜ inch male NPTF quick-connect 27 located at the water outflow port for connection to female end of aqueous-circuit medical devices, a power input 5 for external power source, which allows connection of a power connector which is to be plugged into a wall outlet, and a power toggle on/off switch and/or button 40 that allows the device to be turned on and manually turned off.

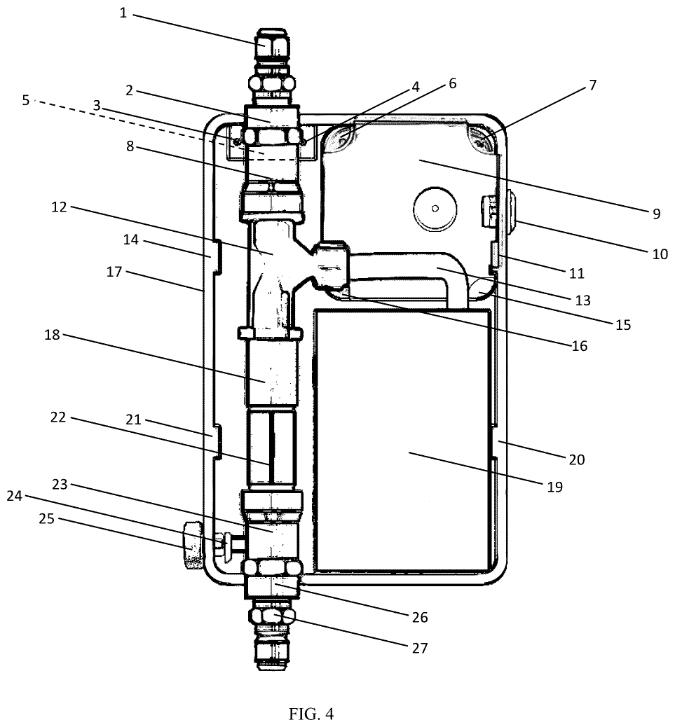

FIG. 4 depicts the two-piece housing of the present invention opened to showcase the internal hose quick connections to the ozone generator and the internal processor for the digital display. As shown in FIG. 4, the housing comprises a Hansen B3-T20⅜ inch male NPTF quick-connect 1 located at the water inflow port for connection to female end of aqueous-circuit medical devices, wherein the connection includes a direct connection of operating room devices such as heater-coolers, a rubber grommet 2 located at the water inflow port between quick-connect male end and device internal water circuit, which acts as a seal between the environment and the device internal while stabilizing the internal circuit, power input screw points 3 and 4, a power input for external power source 5 that connects to a power connector, which is to be plugged into wall outlet, PCB and PCB cover screw points 6, 7, 15, and 16, a ½ inch to ⅜ inch threading converter 8 that connects internal circuit to the external Hansen B3-T20 1 at the point of water inflow, PCB 9 with protective cover that acts as the motherboard for all electronic functions within the device, covered by lid (secured by 6, 7, 15, 16), wherein the PCB 9 controls Power on/off, ozone generation unit, power cord outlet, flow sensor, LED light strip, and alarm located on PCB, a power toggle on/off switch and/or button 10 that allows the device to be turned on and manually turned off, device housing screw point 11, 14, 20, and 21 allowing a short screw to connect top and bottom shell of device housing, a ½ inch venturi injector inline component 12 with built-in one-way valve for aqueous ozone solution, the ½ inch venturi injector inline component 12 uses water flow to create vacuum suction through ¼ inch air inlet, and allows ozone to be ported into internal circuit when there is water flow, a ¼ inch silicon tubing 13 for connecting ozone generator to venturi injection module, an ozone device housing 17 as outer housing designed to hold internal components, a ½ inch female-to-female circuit fitting 18 that connects male threading of venturi injector to male threading of flow sensor, an ozone generator 19 with cooling fan and heat-sink housing, wherein the ozone generator 19 contains corona discharge component for creation of ozone with cooling fan and heat-sink for heat dissipation, a ½ inch turbine flow sensor 22 that sends an electrical signal to the PCB 9 when water flow is active to initiate power to ozone generation unit, ½ inch to ⅜ inch threading converter 23 with ball valve and honeycomb diffusion disk, which is a component that connects internal circuit to external Hansen B3-T20 27 at the point of water outflow while converting both thread size and thread type, wherein the ball valve is to stop flow of water and prevent leaking when disconnecting device from aqueous-circuit medical devices, the honeycomb diffusion disk is to create turbulence within circuit to increase efficiency of ozone dissolution into an aqueous solution, a valve stem with seals 24 that Connects Indicator knob to ball valve located inside thread conversion piece, an indicator knob 25 that allows movements of ball valve from outside of the unit, a rubber grommet 26 located at the water outflow port between quick-connect male end and device internal water circuit, and a Hansen B3-T20⅜ inch male NPTF quick-connect 27 located at the water outflow port for connection to female end of aqueous-circuit medical devices.

The ideal embodiment of the ozone cleaner and connector for aqueous-circuit medical devices is a housing for an ozone generator by way of an outer shell with a screen for output data from the internal device and water metrics. The present invention will have a water inlet and outlet on opposing sides that matches the specifications of the water line connections from the aqueous-circuit medical device it is being used with. It will also have a water inlet and outlet quick connection that will connect directly to the ozone generator with the use of a venturi injection system as needed. The water inlet and outlet on the outer shell will then be attached to the circuit connections of the aqueous medical device. This will be achieved through the connection to the hose lines outside of the device and their quick connect design. This will allow for a continuous circuit design throughout the medical device, the invention, and the ozone generator.

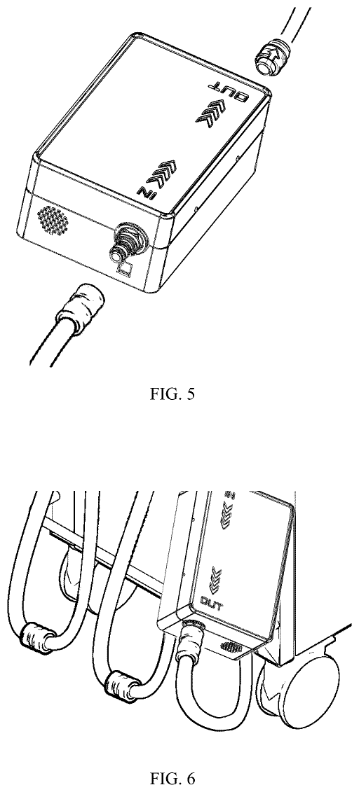

FIG. 5 depicts the quick connection design of male to female hose connections on the ozone cleaner and connector for aqueous-circuit medical devices to be used to connect to the aqueous medical device circuit hose lines.

FIG. 6 depicts one example of an aqueous-circuit medical device with a pair of its hose lines connected to the ozone cleaner and connector for aqueous-circuit medical devices creating a continuous flow throughout the medical device and the aqueous ozone generator housed and connected within the present invention.

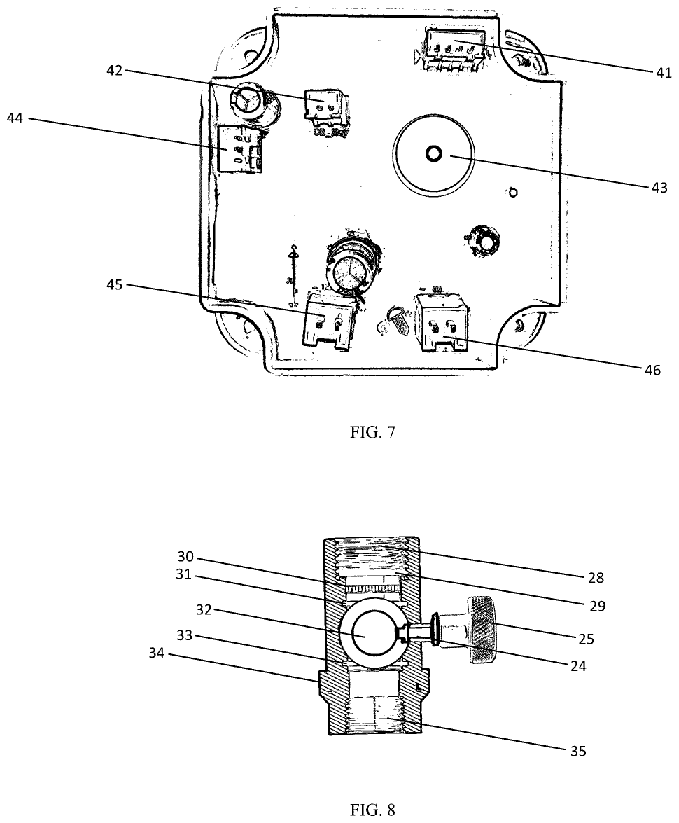

FIG. 7 is a detailed illustration of the PCB comprised in the housing apparatus. The PCB board comprises a LED strip 41 to indicate power on/off and device running status, a power on/off 42, an alarm 43 as an audible indication of power on/off or electrical error, a flow sensor 44, a power cord outlet 45, and an ozone generator controller 46 for controlling the ozone generator 19.

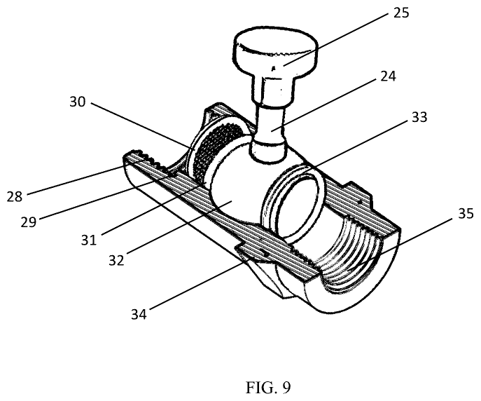

FIGS. 8 and 9 further illustrate components and the constructed structure of the ½ inch to ⅜ inch threading converter 23 with ball valve and honeycomb diffusion disk. The ½ inch to ⅜ inch threading converter 23 further comprises a ½ inch BSP female threading inflow 28 that connects to internal circuit male thread, an O-ring lip 29 inside the ½ inch BSP female threading to create seal and prevent leaking, a honeycomb diffusion disk 30 that creates turbulence and splits air into smaller particles within circuit to increase efficiency of ozone dissolution into an aqueous solution, a ball valve 32 that allows water to flow freely through hole in sphere when in open position and restricts flow of water and leaking when in closed position, a valve housing and gaskets 31 and 33 that create seal and prevent leaking from ball valve, an indicator knob 25 that allows movements of ball valve from outside of the unit, a valve stem with seals 24 as connection between ball valve and indicator knob outside conversion piece, which allows movements of ball valve from open to closed position without leaking, a grommet lock 34 that allows a rubber grommet to be seated on outside of the ⅜ inch section of the conversion piece without unintended movement, a ⅜ inch NPTF threading conversion 35 that connects to external male thread of Hansen B3-T20 quick-connect and acts as both size and thread-type converter from internal to external circuit.

A direct implementation of the ozone cleaner and connector for aqueous-circuit medical devices would be designed to fit into the aqueous-circuit medical devices' internal make-up. This implementation would be achieved by manufacturing the device directly into the medical devices bypassing the need for a separate power supply and instead using connection to medical devices power supply. Depending on the medical device being cleaned, a water pump will be directly integrated into the present invention allowing for water flow throughout the invention and then circulation of aqueous solution within the medical device. This could be implemented through electrolytic technology or by corona discharge, depending on the design of devices needing disinfection.

What is claimed is:

1. A ½ inch to ⅜ inch threading converter for an ozone cleaner for aqueous-circuit medical devices, comprising:
a ½ inch BSP female threading inflow connection configured to connect to an internal circuit male thread of the ozone cleaner;
a ball valve allowing water to flow freely through hole in sphere when the ball valve is in open position and restricts flow of water and leaking when the ball valve is in closed position;
a valve housing and gaskets for creating seal and prevent leaking from the ball valve;
an indicator knob allowing movements of the ball valve from outside of the unit;
a valve stem with seals between the ball valve and the indicator knob outside conversion piece, which allows movements of the ball valve from open to closed position without leaking;
a grommet lock on the outer surface of the ½ inch to ⅜ inch threading converter for allowing a rubber grommet to be seated on; and
a ⅜ inch NPTF threading connection that connects to an external male thread.

2. The ½ inch to ⅜ inch threading converter for an ozone cleaner for aqueous-circuit medical devices according to claim 1, further comprising:
an O-ring lip inside the ½ inch BSP female threading to create seal and prevent leaking; and
a honeycomb diffusion disk for creating turbulence and splitting air into particles within the ozone cleaner to increase efficiency of ozone dissolution into an aqueous solution.

3. The ½ inch to ⅜ inch threading converter for an ozone cleaner for aqueous-circuit medical devices according to claim 2, further comprising:
a grommet lock on the outer surface of the ½ inch to ⅜ inch threading converter for allowing a rubber grommet to be seated on.

4. An ozone cleaner for an aqueous-circuit medical device containing liquid tanks used for patient temperature control, comprising:
a water inflow port;
a water outflow port;
a printed circuit board (PCB);
a ozone generator; and
the ½ inch to ⅜ inch threading converter according to claim 1.

5. The ozone cleaner according to claim 4, further comprising:
a male-to-female connector for connecting the ozone cleaner to an external circuit lines of the aqueous-circuit medical device.

6. The ozone cleaner according to claim 4, further comprising:
tubing and water line connections with male-to-female connectors.

7. The ozone cleaner according to claim 5, wherein the male-to-female connector allows a removeable connection between the ozone cleaner and the aqueous-circuit medical device.

8. The ozone cleaner according to claim 4, wherein the ozone cleaner is mobile and is capable of connecting by way of intersecting water lines in a continuous circuit.

9. The ozone cleaner according to claim 4, wherein the ozone cleaner is connected to a patient incubator for sanitizing and cleaning or disinfecting of internal components and aqueous fluid inside.

10. The ozone cleaner according to claim 4, wherein the water inflow port comprises a Hansen B3-T20⅜ inch male NPTF quick-connect.

11. The ozone cleaner according to claim 10, wherein the water outflow port comprises a Hansen B3-T20⅜ inch male NPTF quick-connect.

12. The ozone cleaner according to claim 4, further comprising:
a rubber grommet located at the water inflow port acting as a seal.

13. The ozone cleaner according to claim 10, further comprising:
a ½ inch to ⅜ inch threading converter connecting an internal circuit of the ozone cleaner to the Hansen B3-T20 connector at the point of water inflow.

14. The ozone cleaner according to claim 4, further comprising:
a ½ inch venturi injector inline component with a built-in one-way valve for aqueous ozone solution, the ½ inch venturi injector inline component uses water flow to create vacuum suction through a ¼ inch air inlet, and allows ozone to be ported into an internal circuit when there is water flow.

15. The ozone cleaner according to claim 4, wherein the PCB further comprises:
a LED strip to indicate power on/off and device running status;
a power on/off;
an alarm as an audible indication of power on/off or electrical error;
a flow sensor;
a power cord outlet; and
an ozone generator controller for controlling the ozone generator.

16. A method for operating the ozone cleaner according to claim 4, comprising:
connecting the ozone cleaner directly to the aqueous-circuit medical device;
turning the aqueous ozone generator on for a designated time to kill bacteria and viruses with aqueous ozone.

17. The method according to claim 16, further comprising:
disconnecting the ozone cleaner from the aqueous-circuit medical device.
